(19) United States
(12) Patent Application Publication
(10) Pub. No.: US 2013/0263864 A1
(43) Pub. Date: Oct. 10, 2013

(54) TEAT DEVICE FOR PREVENTING SNORING AND OTHER HABITS

(71) Applicant: Hubertus von Treuenfels, Eutin (DE)

(72) Inventor: Hubertus von Treuenfels, Eutin (DE)

(21) Appl. No.: 13/819,598

(22) PCT Filed: Aug. 29, 2011
(86) PCT No.: PCT/EP2011/004325

§ 371 Date: Jun. 6, 2013

(30) Foreign Application Priority Data
Aug. 30, 2010 (DE) 20 2010 011 965.0

(51) Int. Cl. A61J 11/00; A61C 5/14; A61F 5/56

(52) U.S. Cl. CPC A61J 11/007; A61F 5/566; A61C 5/14
USPC 128/848; 128/861; 606/234

(57) ABSTRACT

Teat device for preventing habits such as snoring, breathing through the mouth, sucking, grinding and gnashing the teeth, and for training or treating oral functions, said teat device comprising an oral vestibule.

### CROSS-REFERENCE TO RELATED APPLICATIONS

This application is a U.S. National Stage Application which claims priority to PCT/EP2011/004325, filed Aug. 29, 2011, which claims priority to DE 20 2010 011 965.0 filed Aug. 30, 2010.

### STATEMENT REGARDING FEDERALLY SPONSORED RESEARCH

Not applicable

### BACKGROUND OF THE INVENTION

The invention relates to a sucking utensil for preventing habits such as snoring, mouth breathing, sucking, grinding and gnashing teeth, and for the training or therapy of oral functions.

Snoring is the term for breathing-related noise that arises during sleep from fluttering movements of the relaxed soft palate and/or by the sinking back of the tongue and mandible. These are therefore generally associated with an open mouth and mouth breathing. Snoring is frequently dependent on the position of the sleeper. It can also be promoted by restricted nasal breathing, the consumption of alcohol, adiposity, overexertion, etc.

It is known that conventional pacifiers (“dummies”) are suitable for suppressing snoring in many cases. Pacifiers have a rigid oral shield to which a suction body consisting of a soft elastic material is attached. The oral shield is a molded body consisting of a plastic, and the suction body consists of latex or silicone. When used, the oral shield is arranged in front of the lips of the wearer to prevent the pacifier from being swallowed. Arranging the oral shield in front of the lips of the wearer is considered annoying in certain circumstances.

In addition, there are mandibular advancement splints that keep the mandible from sinking back during sleep and hold it in the correct position so that the tongue cannot fall back and promote respiratory noise or even block the respiratory passages.

In addition, a vestibular plate or vestibular splint is known that is placed in the oral vestibule, the area between the lips and anterior teeth, to thereby block mouth breathing. The contours on the inside of the vestibular splint/vestibular plate serve as a toy for the tongue. The tongue is pressed against the anterior teeth and the vestibular splint/vestibular plate by means of the triggered swallowing reflex and is retained there by the arising vacuum. The space within the trachea is expanded, and the vibration of the soft palate is suppressed. This prevents snoring noise. The bioplastic material is rendered even more elastic by being heated in the mouth and individually adapts to the oral vestibule.

U.S. Pat. No. 6,675,804 describes a sucking utensil with a structure having a plurality of lobes for accommodating the tongue, an undulating connector for connecting the cited structure to an inner lip plate and a hollow tube that connects the lip plate to a dome-shaped structure on an outer oral shield. The device is inserted into a use position in the oral cavity so that movement of the tongue is restricted by the structure having the plurality of lobes, and the teeth bite down on the undulating connector, wherein the lip plate is arranged between the teeth and the inner parts of the upper and lower lips. The device aims to normalize breathing during the nighttime. Mouth breathing is promoted by connecting an opening in the lip plate to the atmosphere.

Against this background, the object of the invention is to provide an improved device for preventing habits such as snoring, mouth breathing, sucking, grinding and gnashing teeth, and to provide training/therapy for oral functions.

The object is achieved by the inventive sucking utensil.

The sucking utensil according to the invention for preventing habits such as snoring, mouth breathing, sucking, grinding and gnashing teeth and for providing training/therapy of oral functions comprises an oral vestibular shield that at least partially consists of a flexible flat material adapting to the surface of the teeth and gingiva which is long enough to extend laterally at least to the premolars, and a soft elastic suction body projecting from the inside of the oral vestibular shield and connected at its base to the oral vestibular shield.

The efficacy of the new sucking utensil is derived from the innate and vital sucking reflex. This reaction occurs up to advanced adulthood when an appropriate suction body is placed in the mouth. After all, humans feed themselves by sucking and drinking. Food that is softened and moistened through chewing is sucked by and collected into a bolus on the tongue which conveys it into the throat by swallowing. The suction reflex is triggered when the tongue contacts the suction body or respectively when the mouth is closed by the oral vestibular shield. During the suction process, the rear of the oral cavity closes. As the dorsum of the tongue lifts and the soft palate flexes, the flow of air and hence the fluttering snoring noise through the mouth is suppressed. In contrast to conventional pacifiers, the new sucking utensil offers two significant advantages. The externally visible and hard lip shield is replaced by an oral vestibular shield arranged hidden behind the lips that at least partially consists of a flexible and hence pleasant to wear flat material. The oral vestibular shield adapts to the oral vestibule and does not cause any change in the oral region that is easily identifiable from the outside. It can be designed very thin and, if applicable, even formed as a thin-wall membrane. The thin and flat oral vestibular shield is tolerated by the mucosa, supple and not noticeable from the outside. In particular, it does not generate any arching displacement of the lips and does not have a pacifier-like appearance as is the case with the conventional oral vestibular plate. The design of the sucking utensil is therefore more attractive and is in particular suitable for consideration in relationships.

In addition, it is more effective than a conventional pacifier because snoring is additionally countered by the anterior closure of the mouth. This is secured and reinforced by the lips that enclose the oral vestibular shield as a reaction to contact stimulus and guarantee a sucking effect. The flexible oral vestibular shield consisting of flat material adapts to the surface of the teeth and the gingiva and, in interaction with the saliva and mucosa, seals the mouth to the outside. Consequently, a vacuum arises in the oral cavity during sucking. Once the oral cavity is sealed at the rear by the contacting of the tongue and palate and is closed in the anterior by the contact of the lips and abutment of the oral vestibular shield on the mucosa, the tongue commences its plunger-like movement. A retracted mandible is thereby spontaneously thrust forward together with the tongue and lower lip to achieve the required closure of the mouth (lip contact), the ideal positional relationship between both jaws, and the contact between the tongue and anterior palate. Noisy mouth breathing is hence superseded by quiet nasal breathing.

The effect of the sucking utensil which prevents or at least reduces snoring is based on the double effect of the active posterior closure of the mouth at the oropharynx, and the active anterior closure of in the mouth at the vestibulum. The double effect prevents mouth breathing, including the noisy vibration of the soft palate, by the reactive flexing of the muscles (lips, tongue, neck muscles, throat muscles and many others).

The sucking utensil according to the invention is in general highly suitable for use by people who suffer from obstructive sleep apnea syndrome (OSAS), and whose snoring and sleeping problem is primarily associated with the structural and functional deficits of the oronasal cavity. Furthermore, the sucking utensil can additionally help insomniacs with apnea who suffer from other or respectively additional complaints. This includes diseases such as: Alcoholism, adiposity, allergies, hormonal imbalances, metabolic problems, cardiovascular diseases, etc. In this case as well, the sucking utensil can be effective in conjunction with ENT physicians, internists, psychologists, dentists and other disciplines.

### BRIEF SUMMARY OF THE INVENTION

The oral vestibular shield preferably consists entirely of a flexible flat material. However, the invention also comprises embodiments of the oral vestibular shield that only partially consist of a flexible flat material. In particular, the invention comprises embodiments in which the oral vestibular shield consists of a flexible flat material in the edge areas, and a harder material in a central area. Furthermore, it comprises oral vestibular shields that consist of a flexible flat material in the edge areas, and a harder material in a plurality of inner areas. If the oral vestibular shield only partially consists of a flexible flat material, it can be designed as a single part consisting of a single material, or a plurality of materials. When made only from a single material, flexible and hard areas can be realized by different thicknesses of the material. When made from a plurality of materials, soft and hard areas can be realized by components with a different softness or respectively hardness.

The sucking utensil comprises a soft elastic suction body that projects from the inside of the oral vestibular shield and is connected at its base to the oral vestibular shield. In conjunction with the remaining sucking utensil, the suction body triggers the suction reflex to particularly promote the aforementioned advantageous effects. During sucking, the material of the soft elastic suction body repeatedly stretches toward the rear corresponding to food prepared for swallowing. In addition, the arrangement of the suction body between the teeth which prevents contact between the mandible and maxilla counteracts the grinding and gnashing of teeth.

Similar to the use of a pacifier (“dummy”), the sucking utensil can be used to prevent or cure the habit of sucking fingers (or other objects).

The therapeutic use of special dummies by speech therapists and other therapists to improve respiratory, sucking, swallowing and speech function can also be performed with the sucking utensil according to the invention, perhaps even somewhat more effectively or respectively easily.

According to one embodiment, the suction body has a cavity which is accessible through an opening in the outside of the oral vestibular shield. This particularly yielding design promotes the sucking effect.

According to another embodiment, the suction body has a teat and a neck which is connected at one end to the teat and at the other end to the oral vestibular shield. The teat has a cross-section that is greater in comparison to the neck which reinforces the suction effect. Given its smaller cross section, the neck can be more easily placed between the teeth.

According to another embodiment, the suction body has an orthodontic shape. Orthodontic suction bodies are characterized by a teat having a convex curvature at the top facing the palate, a bevel running to the rear and upward on the bottom facing the tongue, and a concave curvature in the bevel. Alternatively, suction bodies are manufactured with a teat in a round, cherry shape that are, however, less useful to the concept of the invention than suction bodies with an orthodontic shape. Orthodontic suction bodies and suction bodies having a teat with a round, cherry shape are preferably closed at the distal side.

According to one embodiment, the distally closed suction body expands from 20 to 35 mm in the direction of its longitudinal axis. The suction body can be selected within the indicated range depending on age, mouth size or mouth shape.

According to one embodiment, the suction body and/or the oral vestibular shield has at least one cup-shaped recess. The cup-shaped recess is preferably closed and is formed by a concavity in a wall of the suction body and/or the oral vestibular shield. The cup-shaped recess functions as a suction cup which can adhere to the tongue, palate, mucosa or gingiva depending on the position. This embodiment functions in a stimulatory manner on the suction reflex. In addition, it improves and secures the provided adhesion and positioning of the sucking utensil on the one hand and tongue on the other hand. The cup-shaped recess can in particular be a large invagination or trough in a surface of the suction body and/or oral vestibular shield. In the region of the cup-shaped recess, the suction body and/or the oral vestibular shield is elastically designed. The cup-shaped recess can have the same wall thickness as the neighboring regions of the suction body and/or the oral vestibular shield. In addition, the cup-shaped recess can have a lesser wall thickness than the neighboring regions.

According to a preferred embodiment, the suction body has the cup-shaped recess in a surface against which the tongue lies during use, and/or in a surface against which the palate lies during use, and/or the oral vestibular shield has the cup-shaped recess in a surface facing the gingiva and/or lip and buccal mucosa during use. These embodiments enable adhesion by means of suction between the tongue or respectively dorsum of the tongue and the suction body, and/or between the palate and suction body, and/or between the gingiva and/or lip and buccal mucosa and/or oral vestibular shield. The sucking utensil can be provided with one or more cup-shaped recesses at one or at different of the aforementioned positions.

According to one embodiment, the suction body of the sucking utensil has a distal opening. The suction body preferably expands toward the distal opening. The expansion of the suction body towards the distal opening is preferably in the shape of a calyx or trumpet. The distal opening of the suction body serves to accommodate the user's tongue tip. This embodiment functions similarly to the already described cup-shaped recess to stimulate the sucking reflex and advantageously affects the adhesion and positioning of the sucking utensil on the one hand and the tongue on the other hand. In addition, the sucking utensil experiences greater acceptance by users due to the special suction body design. According to a preferred embodiment, the suction body is smooth, and/or the outer edges of the opening are rounded. According to another embodiment, the suction body has a neck and only one section of a teat connected thereto which has the distal opening. The section of the teat preferably corresponds to a proximal section of a teat of an orthodontic suction body or a suction body with a cherry shape without a distal section of the teat.

According to another embodiment, the opening is sealed by means of a sealing part releasably held on the oral vestibular shield. The suction effect generally arises from the suction device having a distal opening in the suction body and an opening of the suction body in the oral vestibular shield even without the sealing part when the user's lips are closed. If the user has difficulty closing his or her lips, the sealing part allows the tongue to still generate a vacuum. The sealing part is releasably held on the oral vestibular shield so that it can be inserted or removed as needed. The sealing part can for example be attachable in the opening of the oral vestibular shield like a pushbutton. The sealing part can have a sealing plate with a pushbutton-like projection that can be pressed into the opening of the oral vestibular shield like a pushbutton. The oral vestibular shield or respectively the suction body can have a channel or groove to receive a bead on the projection. The plate of the sealing part can lie against the outside of the oral vestibular shield in a sealing manner. The plate can consist of a membrane-like flexible material.

According to a preferred embodiment, the suction body with a distal opening expands from 15 to 25 mm in the direction of its longitudinal axis.

According to another embodiment, this suction body has a maximum width of 20 to 30 mm. According to another embodiment, the neck of this suction body has a width of 15 to 25 mm at its narrowest point, and/or a height of 7 to 10 mm.

According to another embodiment, the opening in the oral vestibular shield has a width of 13 to 20 mm and/or a height of 5 to 8 mm.

This information refers to the arrangement of the sucking utensil in the mouth of a user, wherein the width is measured in a horizontal direction and the height is measured in the vertical direction.

According to another embodiment, the oral vestibular shield has a contour adapted to the anatomy of the oral vestibule. The sucking utensil can therefore be comfortably worn, and irritation of the oral mucosa is avoided.

According to one embodiment, the oral vestibular shield follows an elongated contour. The oral vestibular shield is long enough to extend laterally at least to the premolars.

According to another embodiment, the contour of the oral vestibular shield tapers at the gingival edges proximate to the labial frenulum. Irritation of the labial frenulum is avoided by means of these cutouts. According to one embodiment, the oral vestibular shield has V-shaped cutouts in the area of tapering at the top and/or at the bottom.

According to another embodiment, the oral vestibular shield has a rounded contour at the outer edges in the peripheral direction. This yields an additional adaptation to the anatomy of the vestibular fold of the oral vestibule. According to a preferred embodiment, the oral vestibular shield has an edge area that tapers toward the periphery. This makes it particularly soft and supple.

According to a preferred embodiment, the oral vestibular shield has a cat-tongue-shaped (or a lady-finger-shaped) contour. This yields an elongated format that is tapered proximate to the labial frenulum and is rounded at the outer edges.

According to one embodiment, the oral vestibular shield has a lateral maximum extension of 80 to 140 mm, and/or a minimum extension of 15 to 35 mm at the tapering, and/or a maximum extension of 25 to 50 mm at both sides next to the tapering. Given the dimensions of the cited regions, the oral vestibular shield is suitable for wearers of different age categories or mouth shapes or mouth sizes. In addition, the oral vestibular shield can be individually adapted by being cut to size.

An oral vestibular shield that can be worn by many users without being adapted has a lateral maximum extension of 115 to 135 mm, and/or a minimum extension of 15 to 25 mm at the tapering, and/or a maximum extension of 22.5 to 37.5 mm at both sides of the tapering.

The oral vestibular shield made of flexible flat material can be adapted to the respective wearer. For this purpose, the oral vestibular shield can for example be cut to size using scissors or a knife or punch. The material of the oral vestibular shield must be correspondingly selected.

According to one embodiment, the oral vestibular shield has a least one marking line at a distance from the edge of the oral vestibular shield with a path adapted to the anatomy of the oral vestibule and/or the contour of the oral vestibular shield. The marking line can help the wearer adapt the oral vestibular shield by indicating a cutting line along which the oral vestibular shield can be reduced. When the oral vestibular shield has a contour that is adapted to the anatomy of the oral vestibule, the marking line can follow the contour of the oral vestibular shield in parallel. The oral vestibular shield can be provided with a plurality of marking lines to allow the adaption to oral vestibules of a different shape and/or size. The marking line can be a broken line or a continuous line.

According to one embodiment, the oral vestibular shield has at least one bite splint on one side. A sucking utensil with or without a suction body can be provided with a bite splint. The oral vestibular shield can be designed with a single bite splint in the middle that extends in the transverse plane across the incisal ridges of the wearer during use. A plurality such as three bite splints can be arranged in the transverse plane of the oral vestibular shield with or without a suction body. In this case, a bite splint is preferably arranged in each case on different sides of the sagittal plane on the oral vestibular shield, whereas the third bite splint is in the middle. If the sucking utensil does not have a suction body, the middle bite splint can be arranged in the same plane as the two lateral bite splints. When the sucking utensil has a suction body, it can be located above and/or below the suction body.

The suction body and/or the bite splint can be connected to the oral vestibular shield by means of a suitable fastener. The suction body and/or the bite splint is preferably connected as a single part to the oral vestibular shield, for example by adhesion or welding. The suction body and/or the bite splint and oral vestibular shield are preferably manufactured from the start as a single part from a contiguous material.

According to another embodiment, the suction body and/or oral vestibular shield, and/or the bite splint consist of natural rubber (manufactured from latex), and/or silicone, and/or thermoplastic elastomer, and/or bioplastic plastic. The suction body, and/or oral vestibular shield, and/or the bite splint can be manufactured from a single material component, or from a plurality of material components.

In one embodiment, one or more bite splints can be manufactured separately from the sucking utensil from the same or different material and mechanically connected as an addition/expansion to the sucking utensil. This is for example possible by attaching bolts or bars that are connected to the bite splints and have widened sections at one end (such as bolt heads or retention plates) into openings (holes or slots) of the oral vestibular shield. In another embodiment, a plurality of bite splints, either in the middle or on the sides, can be manufactured independent of each other or together as a single-part bite splint element separate from the sucking utensil and from a different material and mechanically linked to the sucking utensil.

It is also possible to provide the suction body, and/or the oral vestibular shield, and/or the bite splint with reinforcement in areas particularly subject to stress, for example a reinforcement strip embedded in the suction body and, if applicable, in the oral vestibular shield. The bite splint can be a mouthguard splint that does not or scarcely deform when the wearer bites down on it. In addition, the bite splint can be designed as a biter splint, wherein it consists of a soft and/or plastically deformable material into which the wearer can bite, at least on the outside against which the wearer bites. In addition, the suction body can have one or more wedge-shaped cutouts (grooves) on the inside that lend greater yielding as, for example, described in WO 2008154968 A1.

The suction body and/or the oral vestibular shield preferably have a wall thickness of 0.5 to 3 mm, and more preferably 1 to 2 mm. The proportions of the bite splint are preferably a wall thickness (height) of 4 to 8 mm, a length of 20 to 30 mm, and a width of 8 to 12 mm. The wall thicknesses of all the components of the sucking utensil (oral vestibular shield, suction body and bite splint) can also be different in deviation from the indicated dimensions depending on the design due to the cited materials.

According to another embodiment, a sucking utensil is manufactured in a dipping process or injection molding process.

The invention further relates to a template of a flat material with a contour and/or marking line adapted to the anatomy of the oral vestibule. The user can first place a template in the oral vestibule and adapt the contour of the oral vestibular shield to the contour of the template if the template is comfortable to wear. If this does not happen at once, the user can cut the template to size, if applicable along the marking line, until it fits well. Then he can use the template as a cutting aid for adapting the oral vestibular shield.

According to another embodiment, the template has an elongated, and/or tapered, and/or rounded contour, and/or marking line at the ends. The template preferably has a cat-tongue-shaped contour and/or marking line. It is also preferable for the template to have different marking lines that make it easier to cut the different contours to size.

The template preferably consists of an inert and moisture-insensitive material. The template preferably consists of a material that feels very similar to the material of the oral vestibular shield. The template preferably consists of the same material as the oral vestibular shield. For example, the template consists of a plastic or plastic film.

Furthermore, the invention relates to a set with a plurality of templates having different contours and/or different marking lines. The user can select the suitable template and then, after testing, the appropriate sucking utensil. The sucking utensil can be offered with oral vestibular shields of different sizes and/or differently shaped oral vestibular shields, wherein a template of the set is assigned to each of the different oral vestibular shields. A coordinated identification of the sucking utensil and the assigned template can make the assignment easier for the user.

Finally, the invention relates to a set having the sucking utensil according to the invention and at least one template according to the invention. The user can select the suitable template or respectively cut it appropriately to size and transfer the contour of the template to the oral vestibular shield of the sucking utensil.

The sucking utensil according to the invention can be designed depending on the indication (grinding, mouth breathing with or without snoring, etc.), and the wearer's acceptance. The sucking utentsil can be designed with or without the suction body, with or without bite splints, with or without suction cups, with suction cups exclusively on the suction body, exclusively on the oral vestibular shield or on the suction body and oral vestibular shield, with or without marking lines and with or without a template. All of the combinations are possible and available to the user or respectively the treating physician as options.

### BRIEF DESCRIPTION OF THE SEVERAL VIEWS OF THE DRAWINGS

The invention will be further explained with reference to the accompanying drawings of exemplary embodiments. In the drawings:

FIG. 1a to e show a front view of a sucking utensil for a child or a wearer with a small mouth size (FIG. 1a), a view from the left side (FIG. 1b), a vertical section from the right (FIG. 1c), a plan view (FIG. 1d) and a perspective view at an angle from above and from the right side (FIG. 1e).

FIG. 2a to e show a front view of a sucking utensil for an adolescent or a wearer with a medium-size mouth size (FIG. 2a), a view from the left side (FIG. 2b), a vertical section from the right (FIG. 2c), a plan view (FIG. 2d) and a perspective view at an angle from above and from the right side (FIG. 2e).

FIG. 3a to e show a front view of a sucking utensil for an adult or a wearer with a large mouth size (FIG. 3a), a view from the left side (FIG. 3b), a vertical section from the right (FIG. 3c), a plan view (FIG. 3d) and a perspective view at an angle from above and from the right side (FIG. 3e).

FIG. 4 shows a sucking utensil in the mouth in a sagittal section of the facial bones of a wearer;

FIG. 5a to e show a front view of a sucking utensil with a mouthguard splint (FIG. 5a), a view from the left side (FIG. 5b), a vertical section from the right (FIG. 5c), a plan view (FIG. 5d) and a perspective view at an angle from above and from the right side (FIG. 5e);

FIG. 6a to e show a front view of a sucking utensil with an oral vestibular shield without a teat (FIG. 6a), a view from the left side (FIG. 6b), a vertical section from the right (FIG. 6c), a plan view (FIG. 6d) and a perspective view at an angle from above and from the right side (FIG. 6e);

FIG. 7a to e show a front view of a sucking utensil with cup-shaped recesses (FIG. 7a), a view from the left side (FIG. 7b), a vertical section from the right (FIG. 7c), a plan view (FIG. 7d) and a perspective view at an angle from above and from the right side (FIG. 7e);

FIG. 8a to f show a front view of a sucking utensil with a cup-shaped recess in the teat and slots for mouthguard splints in the oral vestibular shield (FIG. 8a), a view from the left side (FIG. 8b), a vertical section from the right (FIG. 8c), a plan view (FIG. 8d) and a perspective view at an angle from above and from the right side (FIG. 8e), and the profile of the cup-shaped recess in a sealed section through the sagittal plane (FIG. 8f);

FIG. 9a to f show a front view of a sucking utensil from FIG. 7 with linked, lateral bite splints (FIG. 9a), a view from the left side (FIG. 9b), a vertical section from the right (FIG. 9c), a plan view (FIG. 9d) and a perspective view at an angle from above and from the right side (FIG. 9e), and a side view of a bite splint (FIG. 9f);

FIG. 10a-e show a front view of a sucking utensil with an oral vestibular shield without a teat with slots for bite splints (FIG. 10a), a view from the left side (FIG. 10b), a vertical section from the right (FIG. 10c), a plan view (FIG. 10d) and a perspective view at an angle from above and from the right side (FIG. 10e);

FIG. 11 shows a front view of a template for cutting a sucking utensil to size;

FIG. 12a-g show a front view of a sucking utensil having a suction body with openings in the oral vestibular shield and a distal opening (FIG. 12a), in a view from the left side (FIG. 12b), with a sealing part in a view from the right side (FIG. 12c), with a sealing part in a plan view (12d), with a sealing part in a front view (FIG. 12e), without a sealing part in a perspective view at an angle from above and from the right side (FIG. 12f) and without a sealing part in a plan view (FIG. 12g);

FIG. 13 shows a sucking utensil according to FIG. 12 in the mouth in a sagittal section of the facial bones of a wearer;

FIG. 14 shows a front view of an oral vestibular shield having a contour with a V-shaped tapering.

### DETAILED DESCRIPTION OF THE INVENTION

While this invention may be embodied in many different forms, there are described in detail herein a specific preferred embodiment of the invention. This description is an exemplification of the principles of the invention and is not intended to limit the invention to the particular embodiment illustrated.

In the following explanation, corresponding parts from different exemplary embodiments are provided with the same reference numbers. Parts with the same designation with different designs are identified by a period following the same reference number and then with a subsequent additional number, wherein the following number is different.

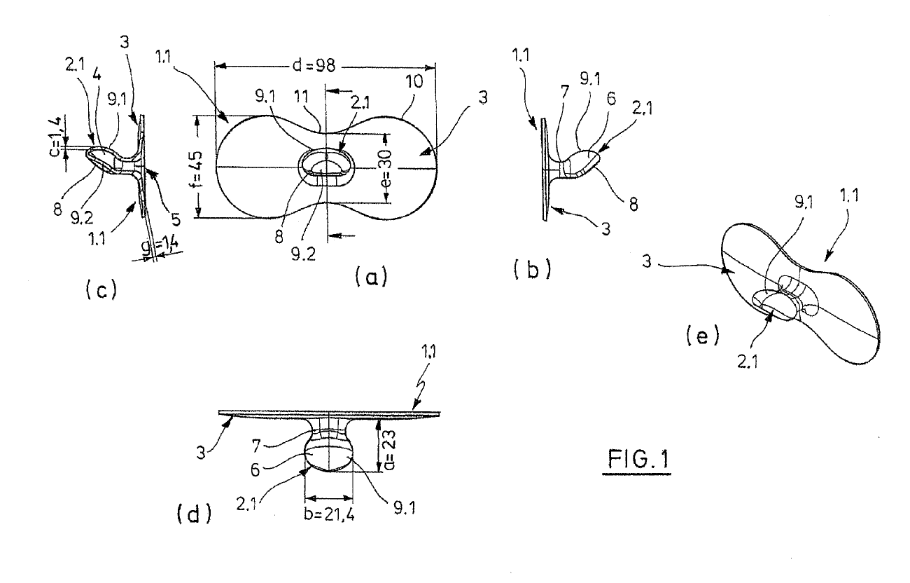

According to FIG. 1, a sucking utensil 1.1 according to the invention has a teat 2 that is connected at the base as a single part to an oral vestibular shield 3.

The teat 2.1 has an interior cavity 4 that is accessible from the outside of the oral vestibular shield 3 through an opening 5 of the oral vestibular shield 3.

The suction body 2.1 has a teat 6 and a neck 7 connected at one end to the teat 6 and at the other end to the oral vestibular shield 3. In the region of the teat 6, the suction body 2.1 is wider than the neck 7 in a transverse plane (i.e., in a plane parallel to the plane of the drawing in FIG. 1d).

At the bottom, the teat 6 has a bevel 8. At the top, it has a convex curvature 9.1 and is rounded in the transition area to the bevel 8. In the center, the bevel 8 has a concave curvature 9.2 or respectively a trough. The neck 7 is rounded in the connecting area to the teat 6 and in the connecting area to the oral vestibular shield 3.

Overall, the suction body 2.1 has an orthodontic shape.

The extension of the suction body 2.1 in the direction of its longitudinal axis or respectively in a sagittal direction is 23 mm. In a transverse direction b, its maximum extension is 21.4 mm. Its wall thickness c is 1.4 mm.

The oral vestibular shield 3 has a cat-tongue-shaped contour 10. In a lateral direction, the oral vestibular shield 3 has an extension d of 98 mm. In the area of the taper 11 of the contour 10, the oral vestibular shield 3 has a minimum extension e of 30 mm. On both sides of the taper 11, the oral vestibular shield 3 has a maximum extension f of 45 mm in each case. The wall thickness g of the oral vestibular shield 3 is 1.4 mm. If applicable, the wall thickness of the oral vestibular shield may vary, for example be thinner toward the periphery and more rigid toward the middle (around the teat opening 5), but not unnecessarily thicker (to support the anterior of the teeth by lip closure or respectively prevent the mandible from retracting).

The sucking utensil 1.1 is manufactured as a single part from latex. Instead, it can be manufactured from silicone or a thermoplastic elastomer. A dipping process is used for manufacturing from latex. Injection molding can be used for manufacturing from silicone or a thermoplastic elastomer. In addition, combinations of materials are possible, wherein the different parts of the sucking utensil can consist of different materials. For example, the oral vestibular shield 3 can consist of a thermoplastic elastomer, and the suction body 2.1 can consist of silicone, wherein the parts can be injected so that they are form-fit to each other. In addition, a safety strip that is integrally connected to the oral vestibular shield 3 and which also consists of the thermoplastic elastomer can be embedded in the suction body 2.1 made of silicone.

The above statements also apply to the following exemplary embodiments to the extent that they are correspondingly designed. In the following, the deviations of the additional exemplary embodiments from the aforementioned exemplary embodiment will be explained in particular.

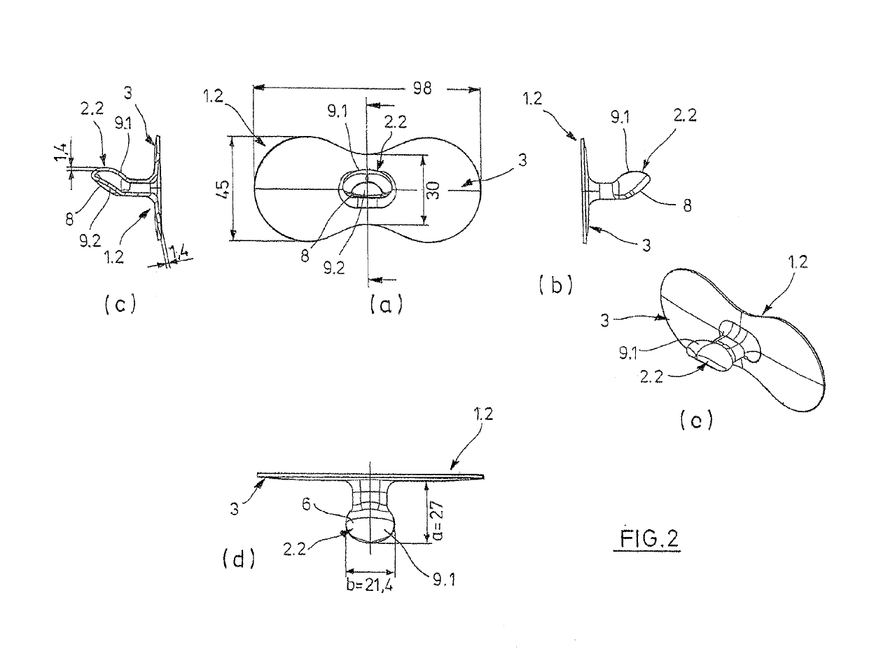

The sucking utensil 1.2 in FIG. 2 differs from the aforementioned in that the extension a of the suction body 2.2 is 27 mm in a longitudinal direction.

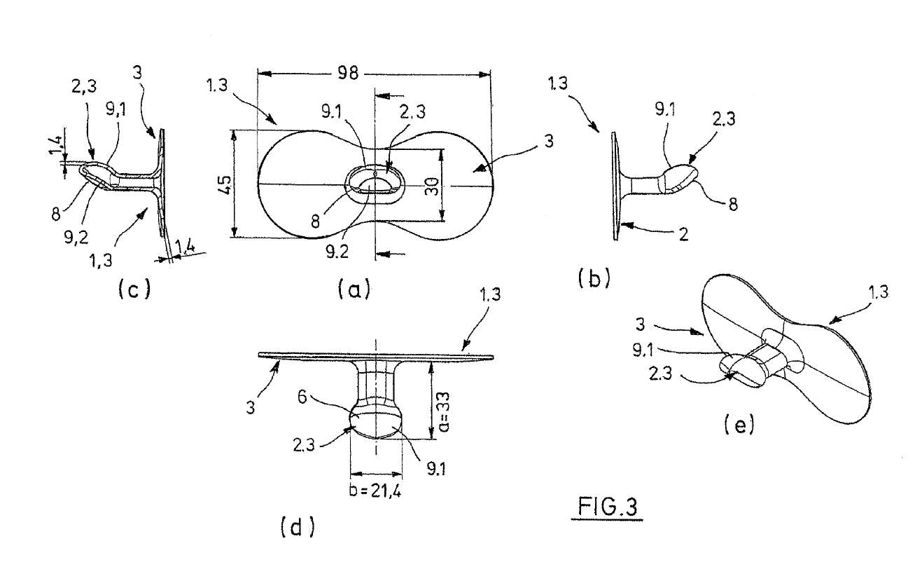

The sucking utensil 1.3 according to FIG. 3 differs from the aforementioned in that the extension a of the suction body 2.3 is 33 mm in its longitudinal direction.

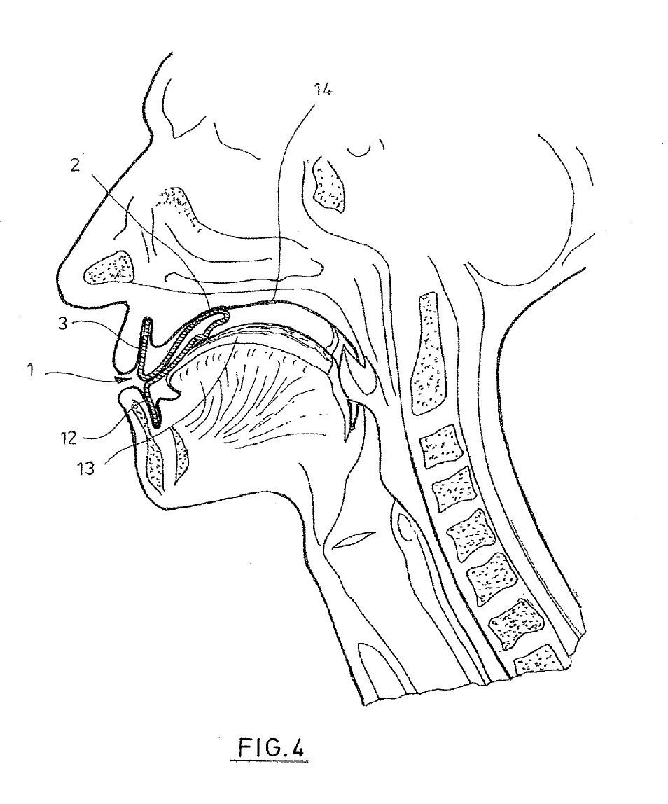

According to FIG. 4, a sucking utensil 1 is arranged in the mouth of a user. The oral vestibular shield 3 is arranged in the oral vestibule 12, and the suction body 2 is arranged between the tongue 13 and palate 14.

The sucking reflex is associated with the lifting and flexing of the rear tongue and the lowering and flexing of the soft palate. Consequently, the mouth is actively closed at the oropharynx. The soft palate closes with the rear dorsum of the tongue to enable nasal breathing and not mouth breathing.

In addition, a reflex-like lip closure effect occurs. The upper lip lowers, and the lower lip raises. The upper lip and lower lip enclose or respectively surround the oral vestibular shield 3. Consequently, the front of the mouth actively closes at the vestibulum.

The front mouth closure and rear mouth closure prevent mouth breathing which includes the noisy vibration of the soft palate (snoring).

Another potential concomitant effect is the tonizising or respectively training of the labial, lingual and pharyngeal muscles. This side effect is based on a learning effect from suction activity so that respiratory noise and/or mouth breathing may be suppressed even without the use of the teat, at least temporarily.

In addition, wearing the sucking utensil 1 causes the retracted mandible to shift forward, and the tongue and lower lip also shift forward. The distance for the required occlusion is thereby improved, that is, the contact of lip to lip, mandible to maxilla, and tongue to palate. Mouth breathing is suppressed in favor of nasal breathing.

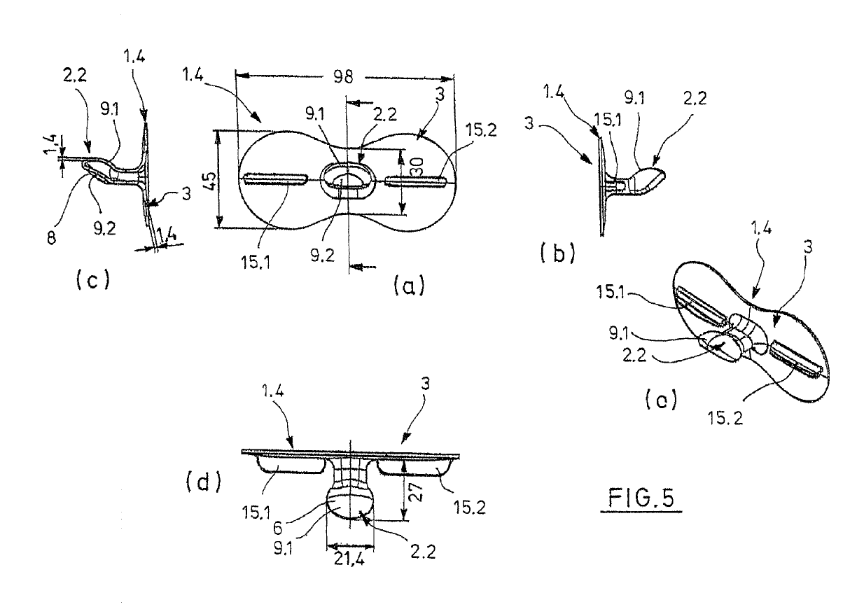

FIG. 5 shows a sucking utensil 1.4 which, in contrast to the sucking utensil 1.2 in FIG. 2, bears a bite splint 15.1, 15.2 on the inside of the oral vestibular shield 3 to either side of the suction body 2.2. The bite splints 15.1, 15.2 are fixedly connected to the sucking utensil 1.2. In this example, the bite splints 15.1, 15.2 are manufactured as a single part with the oral vestibular shield 3 and from the same material as the oral vestibular shield 3. The bite splints 15.1, 15.2 project approximately at a right angle from the oral vestibular shield 3. The bite splints 15.1, 15.2 can also be designed longer or shorter. Furthermore, the sucking utensil 1.4 can also be designed with the dimensions of the sucking utensils 1.1 or 1.3 or other dimensions.

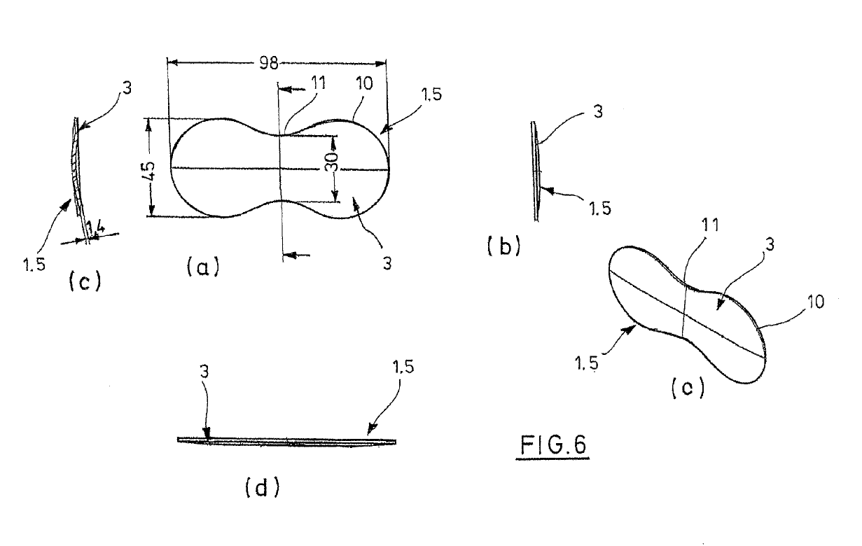

The sucking utensil 1.5 from FIG. 6 differs from the sucking utensil 1.2 from FIG. 2 in that it only has an oral vestibular shield 3 and not a suction body. Optionally, the oral vestibular shield 3 narrows toward the edge areas just like the oral vestibular shield 3 of the sucking utensils 1.1, 1.2 and 1.3. The sucking utensil 1.5 can also be designed with the dimensions of oral vestibular shield 3 of the sucking utensils 1.1 or 1.3 or other dimensions.

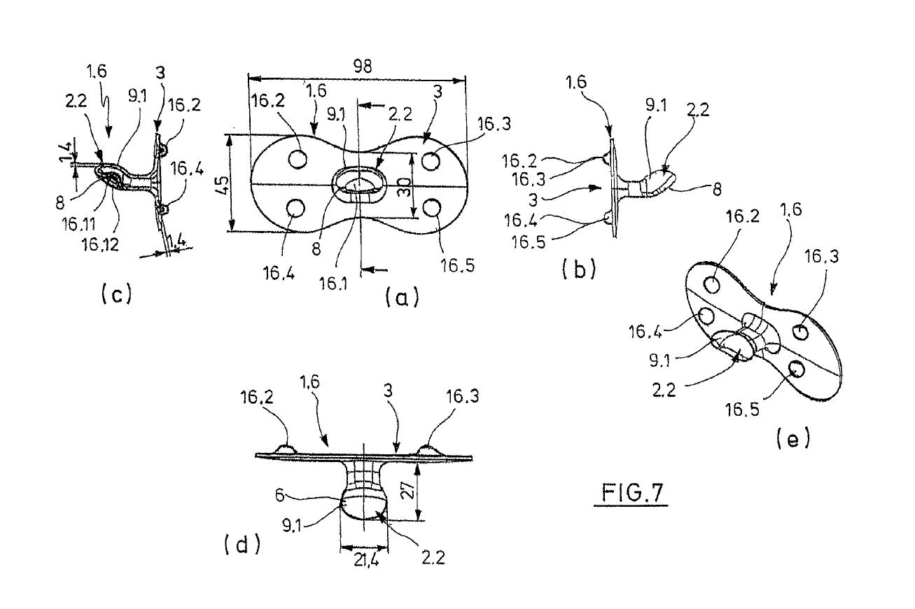

FIG. 7 shows a sucking utensil 1.6 which, in contrast to the sucking utensil 1.2 from FIG. 2, has a cup-shaped recess 16.1 in the center of the bevel 8 of the teat 6 with an opening 16.1,2 in the bevel that functions as a suction cup. The cup-shaped recess 16.1 has a deeper inner curvature in comparison to the concave curvature 9.2 so that, after air is pressed out of the cup-shaped recess 16.1, the suction cup 16.1 can adhere to the dorsum of the tongue.

In addition, the sucking utensil 1.6 has additional cup-shaped recesses 16.2, 16.3, 16.4, 16.5 on the inside of the oral vestibular shield 3 with openings in the inside of the oral vestibular shield 3, wherein the pairs of cup-shaped recesses 16.2, 16.3 and 16.4, 16.5 are each arranged symmetrical to the sagittal plane, and the pairs of cup-shaped recesses 16.2, 16.4 and 16.3, 16.5 are arranged symmetrical to the transverse plane. The suction cups 16.2 to 16.5 can adhere to the gingiva and thereby reinforce the sealed closure of the oral shield. The cup-shaped recess 16.1 also motivates the wearer to play with the teat 6 with the tip of the tongue.

The teat 6 can be provided with a cup shaped recess on the top 9.1 which causes adhesion to the palate.

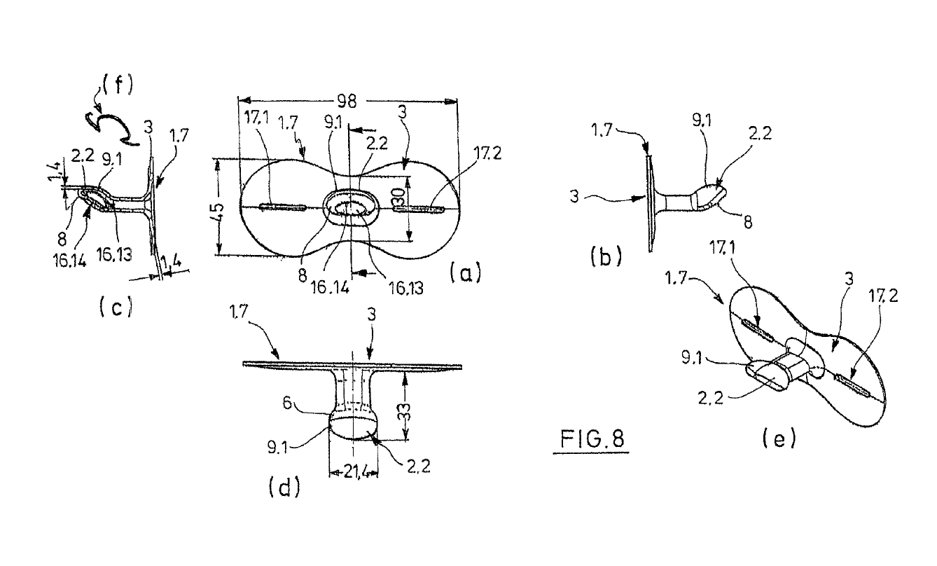

FIG. 8 shows a sucking utensil 1.7 which, in contrast to the sucking utensil 1.6 from FIG. 7, has a more pronounced cup-shaped recess 16.1,2 in the bevel 8 of the teat 6. A sagittal section of the cup-shaped recess 16.1,2 is in the form of an omega and therefore has a smaller cross-section in the area of its opening 16.1.4 in the bevel 8 than in an area located further in the teat 6.

The sucking utensil 1.7 further differs from sucking utensil 1.6 in that the oral vestibular shield 3 has slots 17.1, 17.2 for attaching bite splints on both sides of the suction body 2.2. The user can hence selectively use the sucking utensil 1.7 with or without bite splints. In addition, the cup-shaped recesses 16.2 to 16.5 are missing in the oral vestibular shield 3 of the sucking utensil 1.7 as well as in the top 9.1 of the teat 6 (not shown). An alternative embodiment of the sucking utensil 1.7 which is not shown has cup-shaped recesses 16.2 to 16.5 as well as in the top 9.1 of the teat 6.

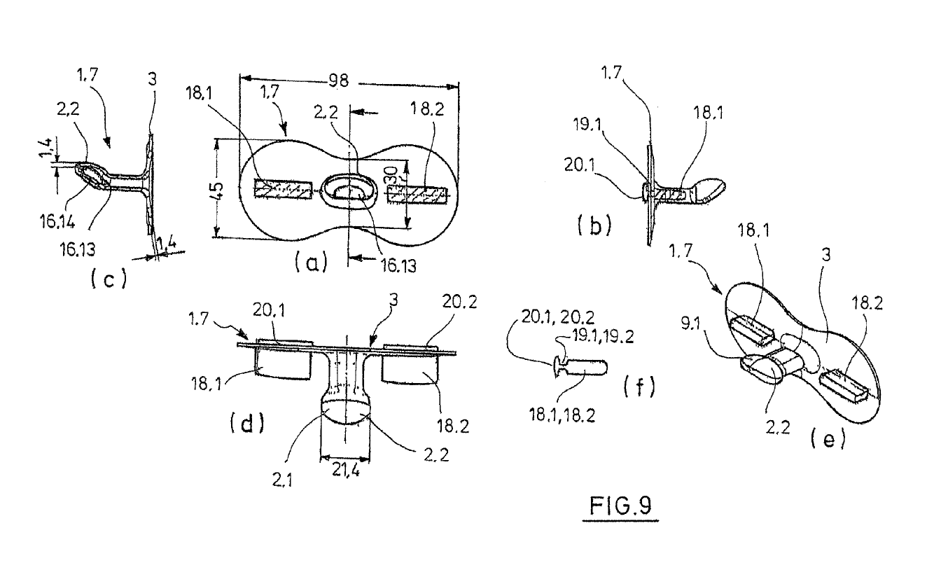

According to FIG. 9, the sucking utensil 1.7 from FIG. 8 is equipped with bite splints 18.1, 18.2. Each bite splint 18.1, 18.2 has a bar 19.1, 19.2 on the face that bears a retention plate 20.1, 20.2 on the end. Instead of the bar 19.1, 19.2, a plurality of pins can also be present on which the retention plate is arranged on the ends, or that are each provided with their own retention plate.

The retention plates 20.1, 20.2 are guided through the slots 17.1, 17.2 in the oral vestibular shield 3 so that the bars 19.1, 19.2 are arranged in the slots 17.1, 17.2, and the retention plates 20.1, 20.2 abut the rear, and the bite splints 18.1, 18.2 rest on the front of the oral vestibular shield 3.

The bite splints 18.1, 18.2 can be biter splints or mouthgard splints. A biter splint can be adapted to the shape of the user's teeth by being bitten into. If applicable, the bite splint 18.1, 18.2 can be designed such that it can be bitten into after being heated for a while (for example in a boiling water bath), and its shape is retained after cooling (in a cold water bath).

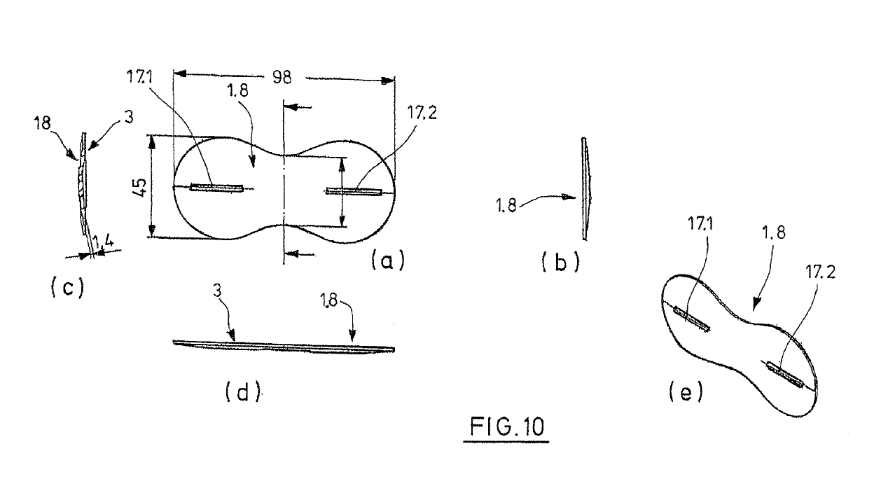

The oral vestibular shield 3 or respectively the sucking utensil 1.8 from FIG. 10 differs from the oral vestibular shield 3 or respectively sucking utensil 1.5 from FIG. 6 in that it has slots 17.1, 17.2 that extend in the transverse plane to either side of the sagittal plane for attaching a bite splint. As needed, the user can use the oral vestibular shield 3 without bite splints or with bite splints. The bite splints 18.1, 18.2 shown in FIG. 9 can be used.

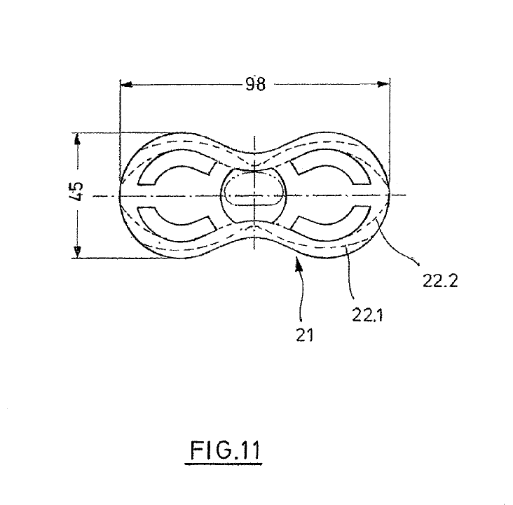

FIG. 11 shows a template 21 that has a cat-tongue-shaped edge contour. In addition, the template 21 has marking lines 22.1, 22.2 of different shapes at a distance from the edge contour.

The template 21 can be placed on an oral vestibular shield 3 that has any original contour. The outer edge of the template 21 or the marking lines 23.1, 23.2 are useful for cutting the oral vestibular shield 3 to size using scissors.

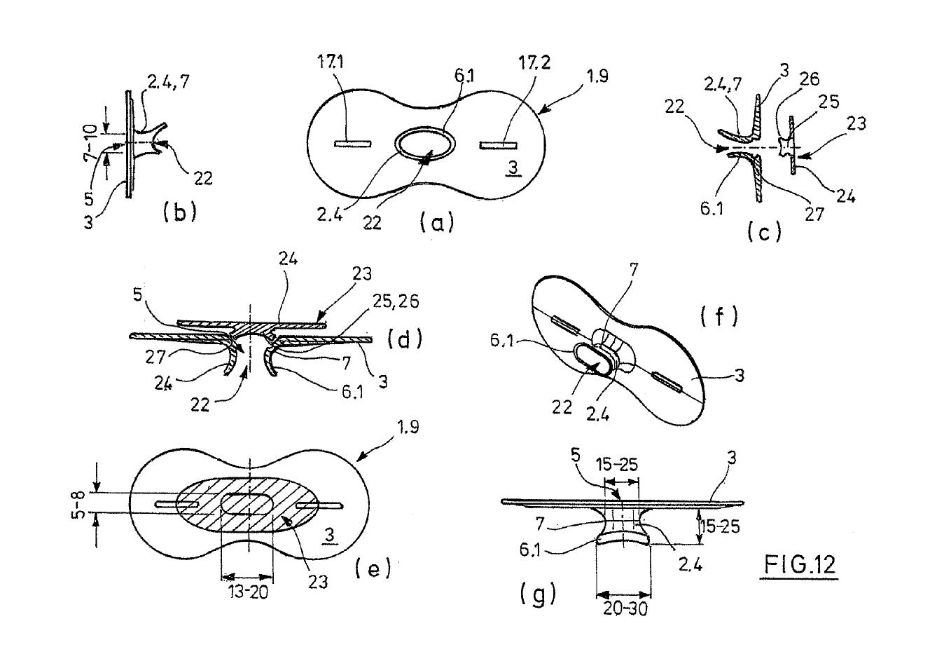

The sucking utensil 1.9 from FIG. 12 differs from the sucking utensil 1.7 from FIG. 8 in that the teat 6.1 does not have the distal part of the teat 6. The suction body 2.4 has a distal opening 22 at that location. In addition, the suction body 2.4 has an opening 5 in the oral vestibular shield 3.

Adjacent to its neck 7, the suction body 2.4 expands toward the distal opening 22 in the form of a calyx. In a horizontal section (in the transverse plane when the sucking utensil is arranged in the mouth of the wearer), the expansion of the cross-section adjacent to the neck 7 is initially stronger, and the expansion is weaker close to the distal opening 22 (FIG. 12g). In a vertical section (in the sagittal plane), the suction body 2.4 expands from the neck 7 to the distal opening 22 more or less uniformly or asymmetrically, wherein the top is preferably curved longer and more strongly than the bottom (FIG. 12b).

A sealing part 23 can be attached in the opening 5 in the oral vestibular shield 3. The sealing part 23 has a sealing plate 24 and a pushbutton 25 which has a radial expansion or respectively bead 26 at the distal end. The sealing part 23 is preferably made of the same material as the sucking utensil 1.9.

The sealing part 23 can be attached in the opening 5. The pushbutton 25 is pressed into the oral vestibular shield 3 and/or the suction body 2.4 which ensures that the sealing part 23 is firmly seated in the suction body 2.4. According to FIG. 12c, the sucking utensil 1.9 can have a peripheral groove, or channel or another recess 27 in the oral vestibular shield 3 and/or suction body 2.4 next to the opening 5, and the radial expansion 26 of the pushbutton 25 can be attached in it.

The sealing part 23 preferably consists of natural rubber, and/or silicone, and/or elastomer, and/or a thermoplastic elastomer.

The sucking utensil 1.9 has slots 17.1, 17.2 in the oral vestibular shield 3, and can be provided with bite splints 18.1, 18.2 like the sucking utensil 1.7 from FIG. 9.

The bite splints preferably consist of a material that can be adapted to the shape of the teeth.

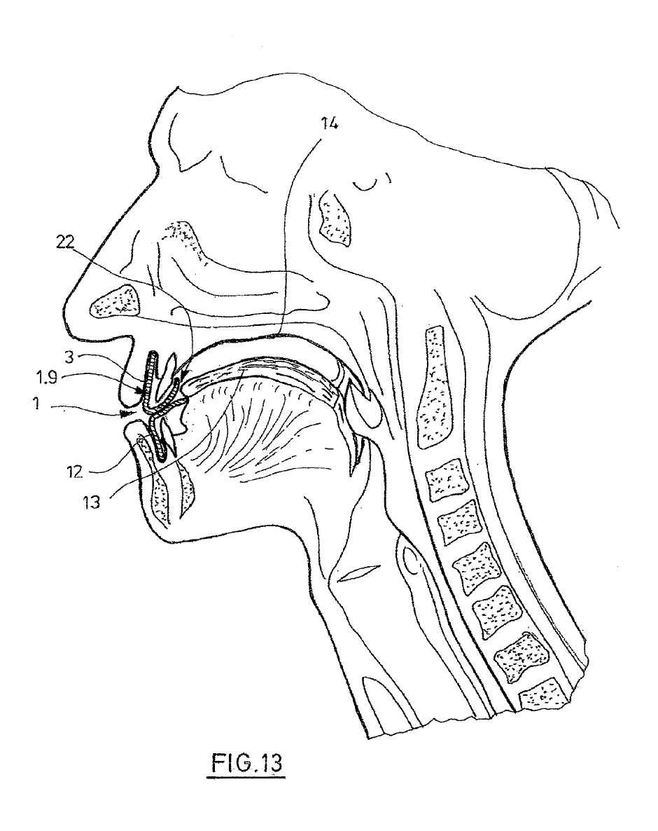

FIG. 13 shows the sucking utensil 1.9 in the mouth of a user. The tip of the tongue 13 engages in the distal opening 22 of the suction body 2.4.

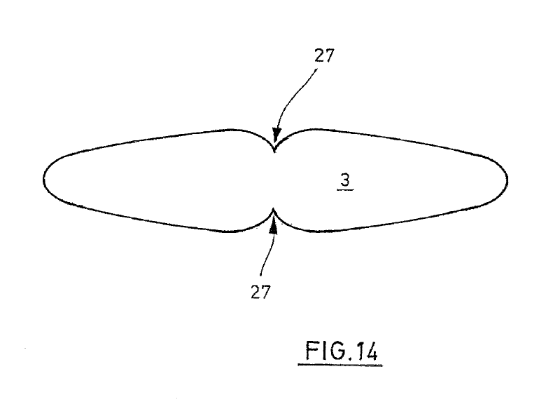

According to FIG. 14, an oral vestibular shield 3 preferably has an elongated contour that narrows at both ends. The middle of the oral vestibular shield 3 has a taper that is formed by V-shaped cutouts 27 in both sides. The oral vestibular shield 3 can be designed in this manner in all exemplary embodiments.

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to”. Those familiar with the art may recognize other equivalents to the specific embodiments described herein which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the preferred and alternate embodiments of the invention. Those skilled in the art may recognize other equivalents to the specific embodiment described herein which equivalents are intended to be encompassed by the claims attached hereto.

1. A sucking utensil for preventing habits such as snoring, mouth breathing, sucking, grinding and gnashing teeth and for providing training or therapy of oral functions comprising an oral vestibular shield (3) that at least partially consists of a flexible flat material.

2. The sucking utensil according to claim 1 having a soft elastic suction body (2) projecting from the inside of the oral vestibular shield (3) and connected at its base to the oral vestibular shield (3).

3. The sucking utensil according to claim 2, wherein the suction body (2) has a cavity (4) that is accessible through an opening (5) in the outside of the oral vestibular shield.

4. The sucking utensil according to claim 2 or 3, wherein the suction body (2) has a teat (6) and a neck (7) connected at one end to the teat (6) and at the other end to the oral vestibular shield (3).

5. The sucking utensil according to one of claims 2 to 4, wherein the suction body (2) has an orthodontic shape.

6. The sucking utensil according to one of claims 1 to 5, wherein the suction body (2) and/or the oral vestibular shield (3) has at least one cup-shaped recess (16).

7. The sucking utensil according to claim 6, wherein the suction body (2) has the cup-shaped recess (16) in a surface against which the tongue rests during use, and/or in a surface against which the palate rests during use, and/or wherein the oral vestibular shield (3) has the cup-shaped recess (16) in a surface that faces the gingiva or buccal mucosa during use.

8. The sucking utensil according to one of claims 2 to 7, wherein the suction body (2) has a distal opening (22).

9. The sucking utensil according to claim 8, wherein the suction body (2) expands toward the distal opening (22).

10. The sucking utensil according to one of claims 8 and 9 as well as 3, wherein the opening (5) in the oral vestibular shield (3) is sealed by means of a sealing part (23) that is releasably held on the sucking utensil (1).

11. The sucking utensil according to one of claims 2 to 10, wherein the distally closed suction body (2) has an expansion of 20 to 35 mm in the direction of its longitudinal axis, and wherein the suction body (2) with a distal opening (22) in the direction of tis its longitudinal axis has an expansion of 15-25 mm.

12. The sucking utensil according to one of claims 1 to 11, wherein the oral vestibular shield (3) has a contour (10) that is adapted to the anatomy of the oral vestibule.

13. The sucking utensil according to claim 12, wherein the oral vestibular shield (3) has an elongated contour.

14. The sucking utensil according to claim 12 or 13, wherein the contour (10) of the oral vestibular shield (3) has a tapering (11) at the gingival margins proximate to the labial frenulum in the maxilla and mandible.

15. The sucking utensil according to one of claims 12 to 14, wherein the oral vestibular shield (3) has a rounded contour (10) over its entire course in the peripheral direction.

16. The sucking utensil according to one of claims 1 to 15, wherein the oral vestibular shield (3) has a cat-tongue-shaped contour.

17. The sucking utensil according to one of claims 12 to 16, wherein the oral vestibular shield (3) has a lateral maximum expansion of 80 to 140 mm, and/or a minimum expansion of 15 to 35 mm at the tapering (11), and/or a maximum expansion of 25 to 50 mm at both sides of the tapering (11).

18. The sucking utensil according to one of claims 1 to 17, wherein the oral vestibular shield (3) has at least one marking line at a distance from the edge of the oral vestibular shield (3) with an outline adapted to the varying anatomy of the oral vestibule and/or the contour of the oral vestibular shield.

19. The sucking utensil according to one of claims 1 to 18 having at least one bite splint (15.1, 15.2) projecting from the inside of the oral vestibular shield (3).

20. The sucking utensil according to claim 19 having a bite splint (15.1, 15.2) that in each case projects from the oral vestibular shield in the region of the anterior teeth and/or in the lateral tooth region.

21. The sucking utensil according to one of claims 2 to 20, wherein the suction body (2) and/or the bite splint (15.1, 15.2) is connected to the oral vestibular shield (3) by means of fasteners or as a single piece.

22. The sucking utensil according to one of claims 1 to 21, wherein the suction body (2), and/or the oral vestibular shield (3), and/or the bite splint (15.1, 15.2) and/or the sealing part consist of natural rubber, and/or silicone, and/or elastomer, and/or thermoplastic material.

23. The sucking utensil according to one of claims 1 to 22 which consists of a plurality of materials.

24. The sucking utensil according to one of claims 1 to 23 that is manufactured in a dipping process or in an injection molding process.

25. A template (21) consisting of a flat material having different contours and/or marking lines (22) adapted to the anatomy of the oral vestibule.

26. The template (21) according to claim 25 with elongated and/or tapered and/or rounded contours and/or marking lines (22) at the ends.

27. A set with a plurality of templates (21) according to claim 25 or 26 having different contours and/or having different marking lines (22).

28. A set with a sucking utensil (1) according to one of claims 1 to 24 and at least one template (22) according to one of claims 25 to 27.
